(19) United States
(12) Patent Application Publication
(10) Pub. No.: US 2010/0030022 A1
(43) Pub. Date: Feb. 4, 2010

(54) METHOD AND SYSTEM WITH ENCAPSULATED IMAGING AND THERAPY DEVICES, COUPLED WITH AN EXTRACORPOREAL IMAGING DEVICE

(76) Inventors: Rainer Graumann, Hochstadt (DE); Rainer Kuth, Hochstadt (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/520,046

(22) PCT Filed: Dec. 19, 2007
(86) PCT No.: PCT/EP07/64215

§ 371 Date: Jun. 18, 2009

(30) Foreign Application Priority Data
Dec. 20, 2006 (DE) 10 2006 060 421.0

(51) Int. Cl. A61B 1/04; G06K 9/00

(52) U.S. Cl. 600/112; 382/128

(57) ABSTRACT

A medical system has an endoscopy system and an extracorporeal imaging system and a patient positioning device. The endoscopy system includes an intracorporeally movable capsule that is navigable within the body of a patient by a magnetic coil system within a tube-like working volume formed by the magnetic coil system. An encapsulated imaging unit in the endoscopy capsule obtains image data associated with a medical finding. The spatial coordinates of the medical finding identified by the encapsulated imaging unit are relayed to the extracorporeal image acquisition system to allow an extracorporeal image to be obtained based on those spatial coordinates.

### BACKGROUND OF THE INVENTION

1. Field of the Invention

The present invention concerns a device for implementation of minimally invasive diagnoses, and when necessary, additional therapeutic procedures inside the body of a patient.

2. Description of the Prior Art

In medicine it is frequently necessary to conduct a medical procedure inside (normally in the body of) a (normally living) person or animal, which medical procedure can be a diagnosis or a treatment, for example. The target of such a medical procedure is often a hollow organ in the patient; in particular the gastrointestinal tract is subjected to such an examination. Such medical measures have been conducted for a long time with the aid of endoscopes which are (invasively or non-invasively) inserted into a patient from the outside, either via bodily orifices of the patient or through small incisions, and are mechanically controlled and positioned.

For catheter-free or tube-less endoscopy, endoscopy capsules that the patient swallows have been known for some years. These capsules are normally provided with image acquisition systems. Endoscopy capsules are known that exclusively passively move forward by peristalsis or can move actively via small drive systems (for example grabbers or propellers).

An endoscopy system composed of an endoscopy capsule and a magnetic coil system is known from DE 101 422 53 C1. This endoscopy system is able to move the endoscopy capsule equipped with a bar magnet in all directions, remotely controlled in the gradient field generated by the magnetic coil system. The force transmission occurs in a targeted manner, without contact and controlled from the outside.

A suitable magnetic coil system that is required in order to move the magnetic endoscopy capsule through hollow organs of a patient by means of magnetic, no-contact force transmission is described in DE 103 40 925 B3. The magnetic coil system is formed by a series of individually controllable individual coils that are arranged so that they fashion a tube-like working space in which the magnetic endoscopy capsule can be moved without contact.

The magnetic coil system from DE 103 41 092 B4 is additionally designed and offers solutions for pausing or floating (hovering) the endoscopy capsule. It is additionally known to equip such endoscopy systems with position detection components that ensure a feedback about the position and possibly also the bearing of the endoscopy capsule inside the body.

These endoscopy capsules possess functionalities of a conventional endoscope; for example, video cameras, small medical instruments or means to administer medicines are integrated into the endoscopy capsule.

As mentioned above, such endoscopy systems of the aforementioned type are used for diagnostics and therapy. However, there are cases in which the diagnostic tools of an endoscopy capsule, just like the diagnostic tools of conventional, mechanical sliding endoscopy, are not sufficient for a complete diagnosis. It is therefore desirable to acquire additional information to generate a diagnosis, for example by external radiological imaging. This can be meaningful to render a pathological finding (that has been initially established by means of the endoscopy) more precisely by acquiring additional data. For example, in this context it can be necessary to determine the penetration depth of the pathological tissue into the healthy tissue in order to be able to establish the size (and therefore the severity) of the illness, and in order to determine suitable therapy measures. Such a task cannot be resolved through the optical image acquisition method of endoscopy. In this case, other and essentially radiological image acquisition methods must be used. Accordingly it would be desirable to be able to immediately conduct a more advanced diagnostic or, if necessary, to be able to immediately derive handling instructions for the continuing endoscopic method, in order to also implement corresponding therapy measures during the endoscopic procedure. A physician may detect a polyp in an endoscopically conducted colonoscopy, which polyp should be removed immediately due to its size, for example, and the removal should likewise appropriately ensue endoscopically and if possible during the same procedure. The greatest danger in the removal of polyps is the possible presence of larger vessels tangential to or contained in that polyp that could lead to more severe bleeding upon a careless removal of the polyp. Although in classical endoscopy an appropriate instrument can be introduced into the operating canal in order to stem the bleeding, this is not possible in the case of capsule endoscopy. Therefore, in the event of a severe bleeding an immediate operation or a procedure with a classical endoscopy system would have to ensue. In this case it is advantageous to know the position of such larger vessels. Such information is to be obtained with external radiological image acquisition systems. In the aforementioned cases, the endoscopic procedure would be interrupted and conducted again upon provision of the missing data. In addition to the interruption of the treatment and the costs therefore incurred for lost time that is caused, an exact assessment using the external image acquisition system is often not possible due to the lack of coordinates of the assessment position. This consequently leads to the situation that the image acquisition region must be selected sufficiently large so that the assessment region of interest is encompassed with high probability. This thus leads to a radiation exposure of more or less large adjacent areas.

Conversely, irregularities or pathologies cannot necessarily be detected with radiological image acquisition systems. Such irregularities or pathologies are diagnosed relatively well with, for example, optical surface-scanning image acquisition systems of endoscopic systems.

### SUMMARY OF THE INVENTION

An object of the invention is to overcome the aforementioned disadvantages and to provide a system that enables an effective, image-supported diagnosis and therapy. The object is achieved by a medical system for image-supported diagnosis or therapy of the body of a patient, possessing

a) an endoscopy system that includes a magnetic coil system and a position detection system, wherein the magnetic coil system controls an endoscopy capsule with at least one image acquisition unit for acquisition of image data of a finding within a tube-like working volume that is formed by the magnetic coil system, which endoscopy capsule can be magnetically navigated in the body of the patient,

b) an image acquisition system with an image acquisition region to acquire image data of a finding outside of the working volume of the magnetic coil system and

c) a patient positioning device,

wherein the endoscopy system and the image acquisition system are functionally and spatially coupled with one another so that the spatial coordinates (finding coordinates) of the finding acquired by the image acquisition unit by means of the position detection system can be relayed to the image acquisition system.

According to the invention, the medical system thus is composed of an endoscopy system and an image acquisition system arranged outside of the body, having a common patient positioning device that ensures that the supported patient is positioned between the working volume of the endoscopy system and the image acquisition region of the external image acquisition system. The position of the endoscopy capsule in the coordinate system of the endoscopy system is known through the position detection system of said endoscopy system. The coordinates of the finding acquired by the image acquisition unit are also known to a sufficient approximation due to the relatively small distance from the endoscopy capsule to the point of the finding. With endoscopy systems that enable a distance measurement between endoscopy capsule and finding subject, this information can be used to correct the finding coordinates since the alignment and support of the endoscopy capsule is also normally known. For the image acquisition system, the position of its image-acquiring components (for example x-ray source and x-ray detector) and the generated image data in the coordinate system of the image acquisition system are described. Endoscopy system and image acquisition system are spatially and functionally coupled. The relationship between the coordinate systems of the endoscopy system and the coordinate system of the image acquisition system is thereby known at all times. Given transmission of the finding coordinates to the image acquisition system, this can thus be adjusted under consideration of the coordinate transformation so that an image acquisition region is focused on the actual finding point. This adjustment of the image acquisition region can ensues automatically or semi-automatically.

In an embodiment of the invention, the spatial coordinates of the finding acquired by the image acquisition system can also be relayed as finding coordinates to the endoscopy system. These can already be coordinates that have been determined via an evaluation of image data of the image acquisition system. Such data generated via evaluation can be depth indicators of detected pathological tissue variations or information about vessel positions, for example. The relayable finding coordinates are not limited to aforementioned application cases. In any case, a more precise positioning of the endoscopy capsule or its invasive instruments is possible in this manner.

In an additional embodiment, the spatial coupling of endoscopy system and image acquisition system is established via the mechanical coupling of both systems. This coupling is achieved in that the systems are arranged fixed on a common base but can be moved relative to one another. A coordinate transformation between the two systems is required only once in this case, and in fact after arrangement at or on the common base. The relative movements between the systems are tapped via suitable position sensor packages, and a continuous updating of the position in the two coordinate systems is possible in computers of the endoscopy and image acquisition system, or of an additional separate computer. Through this functional coupling, every spatial coordinate in the coordinate system of the endoscopy system can be unambiguously mapped to a spatial coordinate of the image acquisition system and vice versa. In this context, it is also conceivable to produce base elements with whose help the mechanical coupling were produced. In this case, endoscopy and image acquisition systems would be individually useable at any time but could also be coupled. If the connection points of the base elements and those of endoscopy system and image acquisition system are executed with one-to-one correspondence, a fixed coordinate transformation stored in the calculation systems of endoscopy system and image acquisition system would be usable in this manner.

In a particular embodiment, the spatial coupling between endoscopy system and image acquisition system is established via a position determination device. Endoscopy system and image acquisition system are not mechanically connected with one another in this case. The cited position determination devices are known from what are known as navigation systems from medical technology. The position determination device consists essentially of at least one sensor that is suitable to conduct the determination of the position and bearing of the image-acquiring components (for example x-ray source and detector) of the image acquisition system and to register its position and bearing change. The sensors of the position determination device can be based on both optically, magnetically, ultrasound-based, radio-based or infrared-based sensor methods. Hybrids of these methods can also be advantageously applied that in combination increase the precision of the position determination of the image-acquiring components or improve the error rate via possible redundant systems. It is thus possible to use a few optical sensors (which normally are CCD cameras) and to supplement these with sensors that, for example, are subject to a magnetic position determination method. It would also be conceivable to supplement optical systems with systems that operate on the basis of measurements of the reflections. If the orientation of the magnetic coil system of the endoscopy system can likewise be adjusted, this is likewise detected by the same or a different position determination device. In this case, multiple cameras can detect the orientation of the endoscopy system and detect relative movements of the image acquisition system and take these into account in a coordinate transformation. When the position determination device is used, corresponding markers are applied to the movable parts. The movement evaluation then ensues by detecting these markers.

In a preferred embodiment, the patient can be freely positioned by the patient positioning device between a working volume and an image acquisition region, and the position change of the patient positioning device can be registered and relayed to the endoscopy system and/or the image acquisition system. In this embodiment, a movement of the patient positioning device can ensue under the assumption that the position of the patient himself remains unchanged, and this movement of the patient positioning device can be taken into account in the image focusing of the image acquisition region on the actual finding point. This is in particular advantageous when the movement of the magnetic coil system of the endoscopy system or the movable components of the image acquisition system that are relevant to the image acquisition are complicated, or are complicated at least in specific movement regions. The patient positioning device should be capable of executing not only a translation position change but also a rotational. The movement changes are detected by measurement sensors and provided to the endoscopy and/or image acquisition system.

In an additional variant, the position change of the patient positioning device can also be detected via a position determination device. The position determination device will advantageously be the same one that is used for the position determination of endoscopy system and image acquisition system. For this purpose the patient positioning device is equipped with markers and is to be calibrated once with the assistance of these markers. In this way separate measurement sensors for translational or rotational movement at the patient positioning device can be foregone.

In a preferred embodiment the image acquisition system radiates x-rays and can be positioned by means of the finding coordinates such that an image acquisition region is focused on the finding. According to this embodiment, the image acquisition system can be controlled based on the known finding coordinates such that exclusively the finding appears in the image acquisition region. The focusing on the finding region is achieved the remote adjustment of x-ray source and detector or, respectively, via adjustment of the available diaphragms at the x-ray source of the image acquisition system. It is also conceivable simultaneously to adapt the intensity of the x-ray radiation to varying distances relative to the acquisition subject. The patient positioning device must be functionally transparent relative to the physical process used by the endoscopy system and the image acquisition system. For example, this can be achieved by making the patient table from an aramid fiber-reinforced plastic.

In an additional embodiment, the transmission of image data of the finding (thus the finding images themselves) between endoscopy system and image acquisition system is also enabled via the coupling of endoscopy system and image acquisition system. The coupling can directly ensue so that the image data in the respective other system are displayed separately or fused with one another. In another case, the coupling can also ensue indirectly via a separate image processing and/or display unit. In this case, endoscopy system and image acquisition system are respectively connected with this common image processing and/or display unit. The image data are transmitted to this unit and there are displayed separately, as previously described, or are presented fused or, respectively, merged with one another. In this way various individual systems are merged into a single system for the purposes of the operator. For example, if the image data of the image acquisition system are 3D image data sets, the fusing of the two image data sets can support the subsequent, continuative endoscopy in that the image generated by means of the image acquisition system is shown in addition to the camera image of the endoscopy capsule. In this way far more precise optical means for orientation are available to the physician who conducts the endoscopy. For example, vessels that were made visible with the image acquisition system by administering contrast agent during the image acquisition are henceforth also made visible to the physician during the endoscopic procedure.

The mechanical system can be managed easily and simply when the endoscopy system and the image acquisition system possess a common operating and display unit, and all operating and display elements are available therewith.

In a further embodiment an acquisition direction for the image acquisition system can be calculated from the finding images that have likewise been transmitted, and the patient positioning device and/or the image acquisition system can be spatially positioned relative to one another to adjust the acquisition direction. The acquisition direction from which the image acquisition unit of the endoscopy capsule has generated image data of the finding is known via the finding coordinates of the endoscopy capsule and the alignment of the endoscopy capsule. The external image acquisition system can in principle acquire this finding region from various angles, and therefore also various acquisition directions. Not all of these conceivable acquisition directions are suitable to acquire an image optimally supporting the diagnosis. For example, bones or organ positions can strongly influence either the achievability or the image acquisition quality. The acquisition direction—thus the direction from which the acquisition is made by means of image acquisition system—can be optimized given knowledge of the general anatomical atlas and/or given knowledge of the finding position. Such an optimization can contain various optimization parameters. For example, an optimization can ensue based on the radiation dose or based on the contrast ratios.

The above object also is achieved in accordance with the present invention by a method for image-supported, intracorporeal endoscopic diagnosis and therapy that includes the steps of introducing an endoscopy capsule intracorporeally into the body of a patient, supporting the patient on a patient positioning device and inserting the patient into a working volume defined by a magnetic coil system, navigating the endoscopy capsule intracorporeally in the working volume by operation of the magnetic coil system, acquiring intracorporeal image data with an encapsulated imaging system in the endoscopy capsule, and establishing coordinates of a medical finding in the image data, relaying the finding coordinates, and possibly the image data, to an extracorporeal image acquisition system located outside of the body of the patient, moving the patient out of the working volume of the magnetic coil system, moving the patient into an image acquisition region of the extracorporeal image acquisition system, aligning the patient and the image acquisition system according to the relayed finding coordinates, and possibly also dependent on the relayed image data, and acquiring an image of at least one further finding location using the extracorporeal image acquisition system.

In one embodiment, the patient is transported into the working volume of the magnetic coil system again after image acquisition via the image acquisition system in order to be able to further treat said patient, possibly under consideration of image exposures of the finding points or finding results automatically or manually derived in the endoscopy system. Such further treatment can contain additional diagnoses or even already include therapy measures.

### BRIEF DESCRIPTION OF THE DRAWINGS

FIG. 1 schematically illustrates a first embodiment of a medical system constructed and operating in accordance with the present invention, wherein coupling of the encapsulated imaging system and the extracorporeal imaging system proceeds mechanically.

FIG. 2 schematically illustrates a second embodiment of a medical system constructed and operating in accordance with the present invention, wherein coupling of the encapsulated imaging system and the extracorporeal imaging system proceeds with a position determination device.

FIG. 3 schematically illustrates an example of use of the endoscopy capsule on the basis of image data obtained with the extracorporeal image acquisition system.

### DESCRIPTION OF THE PREFERRED EMBODIMENTS

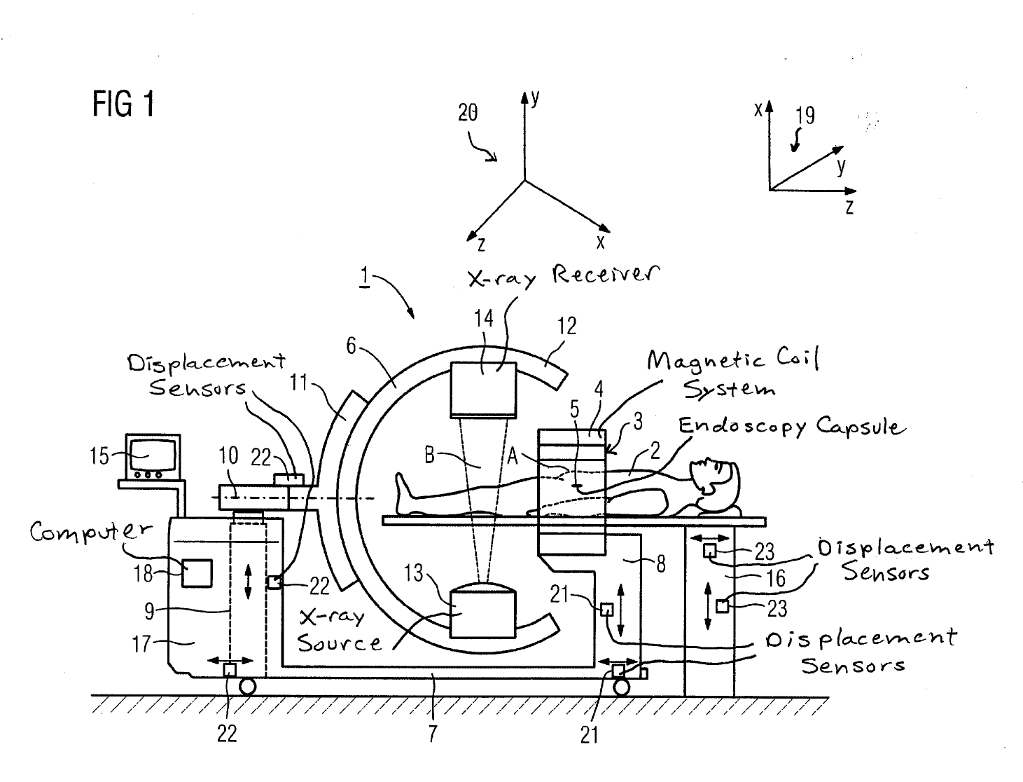

FIG. 1 shows the medical system 1 according to the invention as a mechanically coupled system in which an endoscopy system 3 and an image acquisition apparatus 6 (shown by way of example in the form of a C-arm) are mechanically coupled with one another via a base 7. The endoscopy system 3 essentially is composed of a magnetic coil system 4 and a cylindrical endoscopy capsule 5 that can move freely in a working volume A via the magnetic coil system 4. The working volume A is the space within the magnetic coil system 4 in which the gradient fields generated by the magnetic coil system 4 act on the endoscopy capsule 5. The position and, if applicable, the alignment of the endoscopy capsule 5 in the longitudinal axis are determined via a position detection system (not designated in detail) which is integrated into the magnetic coil system 4. The position detection is mapped in the coordinate system 20 of the endoscopy system 3. The endoscopy capsule 5 is equipped with an image acquisition unit via which image exposures of the inside of the patient 2 are enabled. The image acquisition unit typically includes a CCD camera whose images are sent via radio to a receiver unit. The magnetic coil system 4 is connected via a retention device 8 with a base 7. The retention device 8 can be moved in the vertical and horizontal direction. The displacement in the horizontal or vertical direction can be detected by integrated movement measurement sensors 21. At another point, the base 7 is connected with an additional retention device 9 that is in turn connected at its opposite end with a retention part 10. The retention 9 can likewise be moved in a vertical and horizontal direction. This displacement in the horizontal or vertical direction can also be tapped via integrated movement measurement sensors 22. A fastening device 11 with the C-arm 12 is mounted such that it can rotate on the retention part 10. The displacement of the C-arm can be tapped via an additional movement measurement sensor 22. An x-ray source 13 and an x-ray receiver 14 are mounted opposite one another on the C-arm 12. The x-rays emitted by the x-ray source 13 and striking the x-ray receiver 14 form the image acquisition region B of the x-ray radiation. The x-ray images acquired with the x-ray receiver 14 can be shown in a known manner on a display device 15. The image acquisition system 6 shown in FIG. 1 allows 3D x-ray images to be produced of the body or of body parts of a patient 2 borne on a patient positioning device 16 that can be displaced vertically or horizontally. The vertical and horizontal displacement of the patient positioning device 16 can be measured by means of movement measurement sensors 23. For 3D imaging, in the case of the present exemplary embodiment an image computer 18 arranged in the apparatus cabinet 17 of the medical system 1 and connected (not shown) with the x-ray receiver 14 and the display device 15 is present. The image computer 18 reconstructs the 3D images of the body part of the patient 2 that is to be presented in a coordinate system 19 in a known manner from 2D projections that are acquired given a displacement of the C-arm around the z-axis or, given 2D slice exposures, provides these on the display device 15. The image data of the inside of the body of the patient 2 (acquired via the endoscopy capsule) can additionally be presented on the display device 15. This can be the merged presentation, i.e. superimposed presentation of image data of endoscopy system and image acquisition system.

In the embodiment of FIG. 1, a transformation of position data and image data of the finding between the two systems 3, 6 is possible through the mechanical coupling of the image acquisition system 6 and the endoscopy system 3 via the base 7, together with the measurement sensors 21, 22. Position data of the finding in the endoscopy system 3 can thus serve as desired positions in the coordinate system 19 of the image acquisition system 6 and vice versa (assuming that the medical system remains unmodified in space). However, the position variation of the patient positioning device 16 can be taken into account when its position change can be taken into account in the transformation via the measurement sensor 23. The transformation can be conducted by the image computer 18 or by a separate computer.

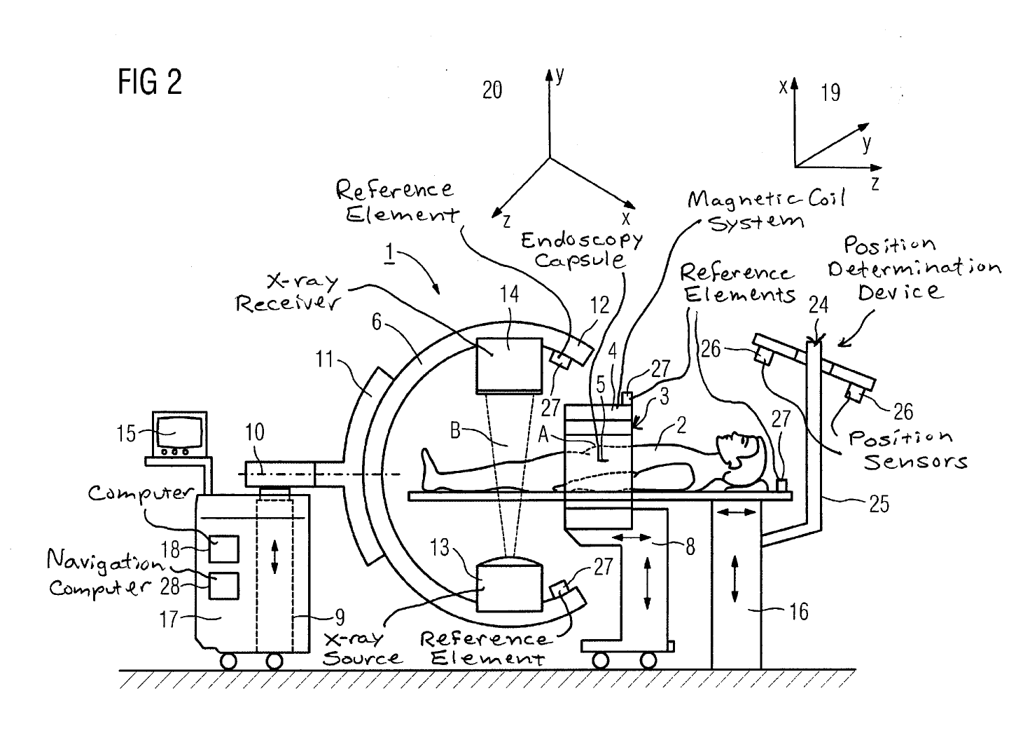

FIG. 2 shows the medical system 1 according to the invention as a functionally coupled system in which an endoscopy system 3, and an image acquisition apparatus 6 (shown in the form of a C-arm, for example) are coupled with one another via a position determination device. In the exemplary embodiment the position determination device 24 is connected with the patient positioning device via a retention arm 25. It can naturally also be attached differently or be set up separately. Position sensors 26 are mounted on the retention arm. In addition to the position sensors 26, the position determination device 24 furthermore comprises reference elements 27 which are associated with the subjects whose position should be detected and are acquired by the position sensors 26. In order to be able to implement a coordinate transformation as described in FIG. 1, the reference elements 27 are arranged on the movable elements of endoscopy system 3 and image acquisition system 27. For example, such reference elements are arranged on the C-arm 12 and at the magnetic coil system 4. At least three of these reference elements 27 are required per system for the coverage of the function and all six degrees of freedom (triangulation). Passive, optical markers with infrared-reflecting surface are advantageously also used today as reference elements since additional, normally disruptive wiring can be foregone with these. A navigation computer 28 likewise belonging to the position determination device evaluates the images acquired with the position determination sensors 26 and can determine the positions (i.e. the bearings and orientations) of the reference elements 27 (and thus of endoscopy system and image acquisition system) in space. The position determination device 24 is to be calibrated in a one-time step. The patient positioning device 16 can also be integrated into the position determination device 24 in order to also take this movement into account. For this purpose the patient positioning device 16 is also to be equipped with reference elements 27.

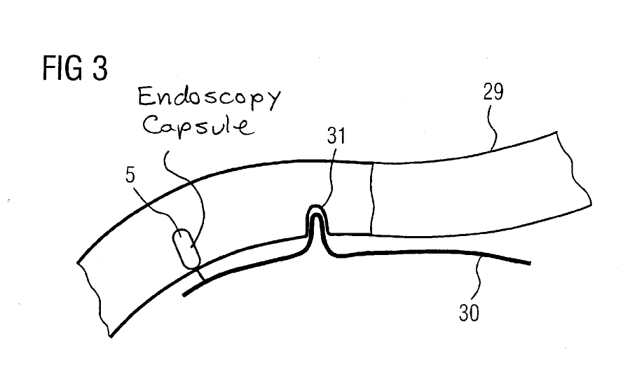

N EXAMPLE OF THE use of an endoscopy capsule 5 on the basis of image data of an image acquisition system is shown in FIG. 3. After lesions have been marked at one or more points by the treating personnel during the endoscopy, these lesions are examined by transfer of the finding position to the image acquisition system via additional (advantageously x-ray) images. The endoscopy is continued according to these image exposures. During the following therapy, a shadow image or slice image or an MPR (multiplanar reconstruction) or an MIP (maximum intensity projection) of the current lesion visible in the endoscopy or accessible to the therapy can advantageously be displayed. FIG. 3 shows a stalked polyp 31 in the colon 29 that is supplied by a larger blood vessel 30. The size and the position of the blood vessel 30 are known due to the images acquired via the image acquisition system 6. An injection implemented magnetically by means of endoscopy capsule 5 is conducted in the further endoscopy, which injection injects a vessel-closing therapeutic agent at a suitable vessel point in order to be able to conduct a subsequent polypectomy with low risk.

Although modifications and changes may be suggested by those skilled in the art, it is the intention of the inventors to embody within the patent warranted heron all changes and modifications as reasonably and properly come within the scope of their contribution to the art.

We claim as our invention:—therefor.

1-13. (canceled)

14. A medical system for image-supported diagnosis and therapy within the body of a patient, comprising:
an endoscopy system comprising an endoscopy capsule, configured for intracorporeal introduction into, and movement within, the body of a patient;
a magnetic coil system that generates a magnetic field that interacts with said endoscopy capsule to guide movement of said endoscopy capsule in the body of the patient, said magnetic coil system defining a tube-like working volume within which said endoscopy capsule is movable by said magnetic coil system;
said endoscopy capsule comprising an encapsulated imaging unit and an encapsulated therapy administration unit, said encapsulated imaging unit acquiring image data representing a medical finding within said tube-like working volume;
an extracorporeal image acquisition system having an image acquisition region in which image data are acquired by the extracorporeal image acquisition system representing a medical finding outside of said working volume of said magnetic coil system;
a coupling device that operationally and spatially couples said endoscopy system and said extracorporeal image acquisition system by correlating spatial coordinates of the finding acquired with said encapsulated image acquisition unit with the image data acquired by said extracorporeal image acquisition system; and
a transmitter in said endoscopy capsule that transmits said image data from said endoscopy capsule in the body of the patient to said extracorporeal image acquisition system.

15. A medical system as claimed in claim 14 wherein said endoscopy system comprises a receiver allowing transmission of spatial coordinates of the medical finding acquired by the extracorporeal image acquisition system to be relayed to said endoscopy system.

16. A medical system as claimed in claim 14 wherein said coupling device mechanically couples said magnetic coil system and said extracorporeal image acquisition system.

17. A medical system as claimed in claim 14 wherein said coupling device is a position determining device configured to determine respective positions of said endoscopy system and said extracorporeal image acquisition system.

18. A medical system as claimed in claim 14 comprising a patient positioning device configured to support said patient thereon and being configured to freely position the patient on the positioning device between the working volume and the image acquisition region, and comprising a processor connected to said patient positioning device that identifies a position change of the patient positioning device, said processor being configured to relay a signal representing said position change to at least one of said endoscopy system and said extracorporeal image acquisition system.

19. A medical system as claimed in claim 19 wherein said patient positioning device comprises a plurality of position-detecting sensors that supply respective signals to said processor, said processor identifying said position change dependent on the signals from said sensors.

20. A medical system as claimed in claim 14 wherein said extracorporeal image acquisition system is an x-ray system that emits x-rays, and comprises a control unit that operates said extracorporeal image acquisition system dependent on the coordinates of the medical finding acquired by said encapsulated imaging unit to orient said image acquisition region relative to said medical finding.

21. A medical system as claimed in claim 14 wherein said coupling device enables exchange of the image data, respectively acquired by said encapsulated image acquisition unit and said extracorporeal image acquisition system, between said endoscopy system and said extracorporeal image acquisition system.

22. A medical system as claimed in claim 21 comprising a processor that merges the respective image data acquired with the encapsulated imaging unit and the image data acquired with the extracorporeal image acquisition system.

23. A medical system as claimed in claim 22 comprising a single display unit connected to said processor at which both image data acquired by said encapsulated imaging unit and image data acquired by said extracorporeal image acquisition system are visually displayed.

24. A medical system as claimed in claim 21 comprising a processor configured to calculate an acquisition direction for said extracorporeal image acquisition system from the coordinates of the medical finding relayed from said encapsulated imaging unit, said processor being configured to operate said extracorporeal image acquisition system to orient said extracorporeal image acquisition system relative to the patient to cause said extracorporeal image acquisition system to acquire image data from said acquisition direction.

25. A medical system as claimed in claim 24 comprising a patient positioning device configured to support the patient thereon, and wherein said processor is configured to operate one or both of said extracorporeal image acquisition system and said patient positioning device to cause said extracorporeal image acquisition system to acquire said image data from said acquisition direction.

26. A method for image-supported endoscopic diagnosis and therapy comprising the steps of:
introducing an endoscopy capsule intracorporeally into the body of a patient;
supporting the patient on a patient positioning device and inserting the patient into a working volume in which the endoscopy capsule is navigable by a magnetic coil system;
navigating the endoscopy capsule in the working volume with said magnetic coil system;
acquiring image data representing a medical finding with an encapsulated imaging unit in the endoscopy capsule, and establishing finding coordinates associated with said medical finding;
relaying the finding coordinates to an extracorporeal imaging system located outside of the body of the patient;
automatically moving the patient out of the working volume of the magnetic coil system and into an image acquisition region of the extracorporeal image acquisition system;
in a processor, calculating an acquisition location and direction using the finding coordinates;
aligning at least one of the patient and the extracorporeal image acquisition system according to said acquisition location and direction; and
acquiring an image of at least one further medical finding using the extracorporeal image acquisition system aligned according to said acquisition location and direction.

27. A method as claimed in claim 26 comprising additionally relaying image data from the image unit in the endoscopy capsule to said processor and, in said processor, calculating said alignment location and direction also using said image data.

28. A method as claimed in claim 26 comprising, after acquisition of said image with said extracorporeal image acquisition system, moving the patient back into said working volume and administering therapy to the patient via said endoscopy capsule in the body of the patient.
